SEQUENCE LISTING (12 sequences)

SEQ ID NO: 1 - DNA, 21 bp, Artificial sequence
  sequence is synthesized
gauaggcgauuaagguuuatt

SEQ ID NO: 2 - DNA, 21 bp, Artificial sequence
  sequence is synthesized
uucagcugugugauccuaggg

SEQ ID NO: 3 - DNA, 21 bp, Artificial sequence
  sequence is snythesized
cgggaaucaaacaagucuatt

SEQ ID NO: 4 - DNA, 21 bp, Artificial sequence
  sequence is synthesized
uagacuuguuugauucccgtt

SEQ ID NO: 5 - DNA, 21 bp, Artificial sequence
  sequence is synthesized
gaaacacgauacuucuugatt

SEQ ID NO: 6 - DNA, 21 bp, Artificial sequence
  sequence is synthesized
ucaagaaguaucguguuuctg

SEQ ID NO: 7 - DNA, 21 bp, Artificial sequence
  sequence is synthesized
gaagaaacuacaacauguatt

SEQ ID NO: 8 - DNA, 21 bp, Artificial sequence
  sequence is synthesized
uacauguuguaguuucuuctg

SEQ ID NO: 9 - DNA, 21 bp, Artificial sequence
  sequence is synthesized
ccugauuuggauacuucuatt

SEQ ID NO: 10 - DNA, 21 bp, Artificial sequence
  sequence is synthesized
uagaaguauccaaaucagggt

SEQ ID NO: 11 - DNA, 21 bp, Artificial sequence
  sequence is synthesized
cuaggaucacacagcugaatt

SEQ ID NO: 12 - RNA, 21 nt, Artificial sequence
  sequence is synthesized
uucagcugugugauccuaggg

(19) United States
(12) Patent Application Publication
(10) Pub. No.: US 2014/0255301 A1
(43) Pub. Date: Sep. 11, 2014

(54) METHODS FOR DETECTING NEURODEGENERATIVE DISEASES OR DISORDERS

(71) Applicants: Mark Chen, New York, NY (US); Ryan J. Watts, San Mateo, CA (US)

(72) Inventors: Mark Chen, New York, NY (US); Ryan J. Watts, San Mateo, CA (US)

(21) Appl. No.: 13/990,747

(22) PCT Filed: Nov. 28, 2011
(86) PCT No.: PCT/US11/62250

§ 371 Date: May 29, 2014

Related U.S. Application Data
(60) Provisional application No. 61/417,701, filed on Nov. 29, 2010.

(51) Int. Cl. G01N 33/68; C12Q 1/68

(52) U.S. Cl. CPC G01N 33/6896; C12Q 1/6883
USPC 424/1.49; 435/6.12; 506/9; 435/7.1; 435/7.92; 435/6.11; 506/16; 506/18

(57) ABSTRACT

Methods of identifying, diagnosing, and prognosing a neurodegenerative disease, or disorder, are provided. Also provided are methods for determining whether a neuron is at risk or is undergoing neurodegeneration. The methods comprise determining whether at least one of the genes tbx6 and dleu2 is overexpressed.

### CROSS REFERENCE TO RELATED APPLICATION

This application claims the benefit of priority of provisional U.S. Application No. 61/417,701 filed Nov. 29, 2010 which is hereby incorporated by reference in its entirety.

### FIELD

Methods of identifying, diagnosing, monitoring and prognosing neurodegenerative diseases or disorders (e.g., Alzheimer's disease) are provided.

### BACKGROUND

The present invention is related to methods and compositions for diagnosis and treatment of a neurodegenerative disease or disorder, such as Alzheimer's disease.

Alzheimer's disease (AD) is a neurodegenerative disorder that results in loss of cognative function and dementia. Ray et al., Nat. Med. 13:1359-1362 (2007). The physical hallmark of AD is the presence of lesions in the brain composed of neurofibrillary tangles (NFTs) and senile plaques which are formed by accumulation of abnormal tau filaments and β-amyloid (Aβ) fibrils. Shaw et al., Nat. Rev. 6:295-303 (2007). The proteins principally responsible for the plaque build up include amyloid precursor protein (APP) and two presenilins (presenilin I and presenilin II). Sequential cleavage of the amyloid precursor protein (APP), which is constitutively expressed and catabolized in most cells, by the enzymes β and γ secretase leads to the release of a 39 to 43 amino acid Aβ peptide. The degradation of APPs likely increases their propensity to aggregate in plaques. It is especially the Aβ (1-42) fragment that has a high propensity of building aggregates due to two very hydrophobic amino acid residues at its C-terminus. The Aβ (1-42) fragment is therefore believed to be mainly involved and responsible for the initiation of neuritic plaque formation in AD and to have, therefore, a high pathological potential. Scientific evidence demonstrates that an increase in the production and accumulation of Aβ protein in plaques leads to nerve cell death, which contributes to the development and progression of AD.

The symptoms of AD manifest slowly and the first symptom may only be mild forgetfulness. In this stage, individuals may forget recent events, activities, the names of familiar people or things and may not be able to solve simple math problems. As the disease progresses, symptoms are more easily noticed and become serious enough to cause people with AD or their family members to seek medical help. Mid-stage symptoms of AD include forgetting how to do simple tasks such as grooming, and problems develop with speaking, understanding, reading, or writing. Later stage AD patients may become anxious or aggressive, may wander away from home and ultimately need total care.

Presently, the only definite way to diagnose AD is to identify plaques and tangles in brain tissue in an autopsy after death of the individual. Therefore, doctors can only make a diagnosis of “possible” or “probable” AD while the person is still alive. Using current methods, physicians can diagnose AD using several tools to diagnose “probable” AD. Physicians ask questions about the person's general health, past medical problems, and the history of any difficulties the person has carrying out daily activities. Behavioral tests of memory, problem solving, attention, counting, and language provide information on cognitive degeneration and medical tests such as tests of blood, urine, or spinal fluid, and brain scans can provide some further information.

It is believed that by the time a patient has been diagnosed with AD, the disease has already been progressing for years. Indeed, understanding the initiation and progression of neurodegenerative disease, such as AD, as well as elucidating mechanisms which underlie or predispose a neuron to degeneration, will aid in the identification of biomarkers to help predict onset, progression and diagnosis of neurodegenerative diseases and disorders. Thus, there remains a need for biomarkers to accurately diagnose neurodegenerative diseases and disorders as well as monitoring progression of the disease and detecting those at risk of developing neurodegenerative diseases and disorders.

All references cited herein, including patent applications and publications, are incorporated by reference in their entirety.

### SUMMARY OF THE INVENTION

The invention provides methods for identifying, diagnosing, monitoring and prognosing a neurodegenerative disorder and/or disorder based at least in part on identification of genes whose expression is associated with neurodegeneration and the presence and/or extent of the neurodegenerative disease or disorder, such as Alzheimer's Disease (AD).

In one aspect, the invention provides a method for diagnosing a neurodegenerative disorder in a subject, the method comprising determining whether a subject comprises a cell that expresses at least one of the genes tbx6 and dleu2 at a level greater than the expression level of the respective genes in a reference sample, wherein the presence of said cell indicates that the subject has said neurodegenerative disorder.

In one aspect, the invention provides a method for monitoring disease in a subject treated for a neurodegenerative disorder, said method comprising determining whether the subject comprises a cell that expresses at least one of the genes tbx6 and dleu2 at a level greater than the expression level of the respective genes in a reference sample, wherein the presence of said cell indicates that the subject is in need of continued treatment for said neurodegenerative disorder.

In one aspect, the invention provides a method for assessing predisposition of a subject to develop a neurodegenerative disorder, said method comprising determining whether the subject comprises a cell that expresses at least one of the genes tbx6 and dleu2 at a level greater than the expression level of the respective genes in a reference sample, wherein the presence of said cell is indicative of a predisposition for the subject to develop a neurodegenerative disorder.

In one aspect, the invention provides a method of determining whether a neuron is at risk and/or is undergoing neuronal degeneration comprising determining whether the neuron expresses at least one of the genes tbx6 and dleu2 at a level greater than the expression level of the respective gene in a neuron not undergoing neuronal degeneration, wherein the increased expression of at least one of the genes tbx6 and dleu2 indicates that the neuron is at risk and/or is undergoing neuronal degeneration.

As would be evident to one skilled in the art, in any method of the invention, while detection of increased expression of a gene would positively indicate a characteristic of a neurodegenerative disorder (e.g. presence, stage or extent), non-detection of increased expression of a gene would also be informative by providing the reciprocal characterization of the disease.

In one aspect of the invention, the neurodegenerative disease or disorder is Alzheimer's Disease (AD), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), Adult Onset Diabetes, senile cardiac amyloidosis, endocrine tumors, glaucoma, Alexander disease, Alper's disease, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Huntington disease, Kennedy's disease, Krabbe disease, Machado-Joseph disease (Spinocerebellar ataxia type 3), Multiple System Atrophy, Neuroborreliosis, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Charcot-Marie-Tooth disease, Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, Islets of Langerhans diabetes Type II insulinoma and other amyloidosis-related diseases.

In one aspect the invention provides a method wherein determining whether the subject comprises a cell that expresses at least one of the genes tbx6 and dleu2 at a level greater than the expression level of the respective genes in a reference sample comprises determining the RNA and/or protein expression levels for at least one of the genes tbx6 and dleu2. In certain examples of the invention, tbx6 expression is determined based on protein expression or RNA expression levels and dleu2 expression is determined based on RNA expression levels. In other examples of the invention the expression of levels of both tbx6 and dleu2 are determined.

In one aspect the invention provides a method wherein determining whether the subject comprises a cell that expresses at least one of the genes tbx6 and dleu2 at a level greater than the expression level of the respective genes in a reference sample further comprises obtaining a biological sample from the subject. In certain examples of the invention, the biological sample is selected from the group consisting of blood, including whole blood, plasma or serum, urine, cerebrospinal fluid, brain tissue (e.g., biopsy) tears and saliva.

In another aspect, the invention provides a method wherein determining whether the subject comprises a cell that expresses at least one of the genes tbx6 and dleu2 at a level greater than the expression level of the respective genes in a reference sample is performed in vivo and does not require obtaining a biological sample from the subject. For example, the method can comprise administering a detectable quantity or effective amount of a labeled probe to the subject and detecting the expression of at least one of the genes tbx6 and dleu2.

The step in the methods of the present invention for determining whether the subject comprises a cell that expresses at least one of the genes tbx6 and dleu2 at a level greater than the expression level of the respective genes in a reference sample may be conducted in a variety of in vitro assays formats including, but not limited to, assays detecting RNA expression or immunohistochemistry assays. In certain examples of the invention the expression of at least one of the genes tbx6 and dleu2 is determined using a PCR method, microarray chip or an immunoassay (e.g. ELISA), or a combination of methods.

The step in the methods of the present invention for determining whether the subject comprises a cell that expresses at least one of the genes tbx6 and dleu2 at a level greater than the expression level of the respective genes in a reference sample in vivo, without obtaining a biological sample, may be determined using a variety of imaging methods including, but not limited to gamma imaging, magnetic resonance imaging (MRI), magnetic resonance spectroscopy, fluorescence spectroscopy, positron emission tomography (PET), single photon emission tomography (SPECT), x-ray computed tomography (CT), fluorescence-mediated molecular tomography (FMT), fluorescence reflectance imaging (FRI), bioluminescence imaging (BLI).

The step in the methods of the present invention for determining whether the subject comprises a cell that expresses at least one of the genes tbx6 and dleu2 at a level greater than the expression level of the respective genes in a reference sample may be determined with the use of a labeled probe. Probes for use in the methods of the invention include, but are not limited to polynucleotides, antibodies or a combination thereof. In certain aspects of the invention, the polynucleotide probes are antisense polynucleotides and/or peptide nucleic acid (PNA) probes. Antibody probes for use in the methods of the invention include, but are not limited to, monoclonal antibodies, chimeric antibodies, humanized antibodies, Fv fragments, Fab fragments, Fab′ fragments, and F(ab′)2 fragments.

In other aspects of the methods of the present invention, the probe for use in the methods of the present invention is conjugated to a brain targeting peptide. In certain aspects the brain targeting peptide allows transport across the blood brain barrier (BBB) via carrier-mediated transport or receptor-mediated transocytosis. Examples of such brain targeting peptides include but are not limited to insulin, transferrin or receptor specific peptidomimetic antibodies which bind to transport receptors on the blood brain barrier (BBB) such as insulin receptor, transferrin receptor, leptin receptor, GLUT1 glucose transporter, MCT1 lactate transporter, LAT1 large neutral amino acid transporter, and CNT2 adenosine transporter.

Probes for use in the methods of the invention may comprise a label, for example, radionuclides, radioisotopes or isotopes and fluorescent dyes as described herein. The label may be, incorporated, attached or conjugated to the probe.

In another embodiment, the invention provides a kit comprising labeled probes for detecting expression of at least one of the genes tbx6 and dleu2 and instructions for using the probes to determine whether a subject comprises a cell that expresses at least one of the genes tbx6 and dleu2 at a level greater than the expression level of the respective genes in a normal reference sample. In certain embodiments the kit is for diagnosing, monitoring, and/or assessing a predisposition for a subject to develop a neurodegenerative disease and/or disorder. In other embodiments, the kit is for determining whether a neuron is at risk and/or is undergoing neurodegeneration.

In other embodiments, the kit comprises labeled probes selected from the group consisting of polynucleotides, antibodies or a combination thereof. In certain aspects, the antibody probes are selected from the group consisting of a monoclonal antibody, a chimeric antibody, a humanized antibody, a Fv fragment, a Fab fragment, a Fab′ fragment, and a F(ab′)2 fragment. In other aspects the probe is an antisense polynucleotide or a peptide nucleic acid (PNA). In other aspects, the probe is labeled and/or conjugated to a brain targeting peptide as described herein.

### BRIEF DESCRIPTION OF THE DRAWINGS

FIG. 1 is a schematic diagram of the experiments performed, as described in Example 1, which use a Campenot chamber, in which somal (cell body) and axonal environments of the neuron are separated.

FIG. 2 is a graph showing the results of experiments as described in Example 1. Specifically, neurons were cultured in Campenot chambers in which the cell body portion contained NGF in the presence or absence of inhibitor and the axonal portion was subjected to NGF withdrawal in the presence or absence of inhibitor. The following inhibitors were used: epidermal growth factor receptor kinase inhibitor AG555 (ErbB^{AG555}); the p38 MAP kinase inhibitor SB239 (p38MAPK^{SB239}); a transcription inhibitor actinomycin D (Transcription^{ActD}); and the GSK3 inhibitor SB415 (GSK3^{SB415}). As can be seen in FIG. 2, actinomycin D and SB415 both prevented axonal degeneration when applied to the cell body portion of the neuron (cell body inhibition), but did not provide the same protective effect when applied directly to the axon (axon inhibition). Additionally, AG555 and SB239 when applied directly to the axon prevented axonal degradation but did not provide the same protective effect when applied directly to the cell body.

FIG. 3 shows the results of a time course microarray experiment on neurons selectively undergoing axon loss. The top panel is a schematic diagram of the experiment as described in Example 2. Both dleu2 and tbx6 are upregulated in neurons experiencing axonal degeneration by 12 hours. Increased expression of dleu2 and tbx6 is not observed in neurons cultured in the presence of the GSK3 inhibitor GSK3.ARA.

FIG. 4 depicts the results of dleu2 and tbx6 knockdown experiments, as described in Example 3. Knockdown of both genes in neurons resulted in reduced axonal degeneration after NGF withdrawal.

FIG. 5 depicts the results of dleu2 and tbx6 knockdown experiments, as described in Example 3. Knockdown of both genes in neurons resulted in reduced axonal degeneration in the presence of a constitutively active GSK3 mutant, GSK3S9A.

FIG. 6 is a plot of tbx6 and dleu2 expression in the hippocampus portions of the brain from human subjects which have been diagnosed with Alzheimer's disease (AD) compared to normal human patients.

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The inventors have discovered biochemical markers useful for the diagnosis of a neurodegenerative disease or disorder, prognosing a neurodegenerative disease or disorder and monitoring a neurodegenerative disease or disorder in a subject (e.g., tracking disease progression in AD patients, which may be useful for tracking the effect of medical therapy in AD patients). Additionally, the biochemical markers are useful for the identification of neurons at risk of undergoing neurodegeneration. The biomarkers for use in the methods of the invention are present in patient biological samples, for example, blood, cerebrospinal fluid, and/or brain tissue.

### CERTAIN DEFINITIONS

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to single-stranded, synthetic polynucleotides that are generally, but not necessarily, less than about 250 nucleotides in length. The terms “oligonucleotide” and “polynucleotide” are not mutually exclusive. The description above for polynucleotides is equally and fully applicable to oligonucleotides.

The term “primer” is generally a short, single stranded polynucleotide that is capable of hybridizing to a nucleic acid and allowing the polymerization of a complementary nucleic acid, generally by providing a free 3′-OH group.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., PCR). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “detection” includes any means of detecting, including direct and indirect detection.

“Elevated expression” or “elevated levels” refers to an increased expression of an mRNA or a protein in a patient relative to a control, such as an individual or individuals who are not suffering from the neurodegenerative disorder and/or a predetermined threshold level.

Expression/amount of a gene or biomarker in a subject or in a first sample (e.g. a biological sample obtained from a subject) is at a level “greater than” the level in a second sample (e.g. a control sample or reference sample) if the expression level/amount of the gene or biomarker in the subject or first sample is at least about 1.5×, 1.75×, 2×, 3×, 4×, 5×, 6×, 7×, 8×, 9×, or 10× the expression level/amount of the gene or biomarker in the second sample.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 C; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55 C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “biomarker” or “biochemical marker” as used herein refers generally to a molecule, including a gene, protein, carbohydrate structure, or glycolipid, the expression of which in or on a mammalian tissue or cell can be detected by standard methods (or methods disclosed herein) and is predictive, diagnostic and/or prognostic for a mammalian cell's or tissue's sensitivity to neurodegeneration. Additionally, a “biomarker” as used herein refers to an indicator of, e.g. a pathological state of a patient, which can be detected in vitro or in vivo in the subject or in a biological sample obtained from the subject.

The terms “neurodegenerative disease” and “neurodegenerative disorder” are used in the broadest sense to include all disorders the pathology of which involves neuronal degeneration and/or dysfunction, including, without limitation, peripheral neuropathies, motorneuron disorders, such as amyotrophic lateral sclerosis (ALS, Lou Gehrig's disease), Bell's palsy, and various conditions involving spinal muscular atrophy or paralysis; and other human neurodegenerative diseases, such as Alzheimer's Disease (AD), Lewy body dementia, Down's syndrome, hereditary cerebral hemorrhage with amyloidosis (Dutch type); the Guam Parkinson-Dementia complex, progressive supranuclear palsy, multiple sclerosis, epilepsy, Creutzfeld Jacob disease, nerve deafness, Meniere's disease, Parkinson's disease, HIV-related dementia, Adult Onset Diabetes, senile cardiac amyloidosis, endocrine tumors, glaucoma, Alexander disease, Alper's disease, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Huntington disease, Kennedy's disease, Krabbe disease, Machado-Joseph disease (Spinocerebellar ataxia type 3), Multiple System Atrophy, Neuroborreliosis, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Charcot-Marie-Tooth disease, Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, Islets of Langerhans diabetes Type II insulinoma and other amyloidosis-related diseases.

“Peripheral neuropathy” is a neurodegenerative disorder that affects the peripheral nerves, most often manifested as one or a combination of motor, sensory, sensorimotor, or autonomic dysfunction. Peripheral neuropathies may, for example, be genetically acquired, can result from a systemic disease, or can be induced by a toxic agent, such as a neurotoxic drug, e. g. antineoplastic agent, or industrial or environmental pollutant. “Peripheral sensory neuropathy” is characterized by the degeneration of peripheral sensory neurons, which may be idiopathic, may occur, for example, as a consequence of diabetes (diabetic neuropathy), cytostatic drug therapy in cancer (e.g. treatment with chemotherapeutic agents such as vincristine, cisplatin, methotrexate, 3′-azido-3′-deoxythymidine, or taxanes, e.g. paclitaxel [TAXOL®, Bristol-Myers Squibb Oncology, Princeton, N.J.] and doxetaxel [TAXOTERE®, Rhone-Poulenc Rorer, Antony, France]), alcoholism, acquired immunodeficiency syndrome (AIDS), or genetic predisposition. Genetically acquired peripheral neuropathies include, for example, Refsum's disease, Krabbe's disease, Metachromatic leukodystrophy, Fabry's disease, Dejerine-Sottas syndrome, Abetalipoproteinemia, and Charcot-Marie-Tooth (CMT) Disease (also known as Proneal Muscular Atrophy or Hereditary Motor Sensory Neuropathy (HMSN)). Most types of peripheral neuropathy develop slowly, over the course of several months or years. In clinical practice such neuropathies are called chronic. Sometimes a peripheral neuropathy develops rapidly, over the course of a few days, and is referred to as acute. Peripheral neuropathy usually affects sensory and motor nerves together so as to cause a mixed sensory and motor neuropathy, but pure sensory and pure motor neuropathy are also known.

The term “diagnosis” is used herein to refer to the identification or classification of a molecular or pathological state, disease or condition such as the identification of a neurodegenerative disorder, e.g. AD.

The term “prognosis” is used herein to refer to the prediction of the likelihood of a neurodegenerative disorder-attributable disease symptom. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely.

As used herein, “treatment” refers to clinical intervention in an attempt to alter the natural course of the individual or cell being treated, and can be performed before or during the course of clinical pathology. Desirable effects of treatment include preventing the occurrence or recurrence of disease symptoms, diminishing any direct or indirect pathological consequences of the disease, decreasing the rate of disease progression, ameliorating or palliating the disease state, and remission or improved prognosis. In some embodiments, methods of the invention are useful in attempts to delay development of a disease or disorder.

An “effective amount” refers to an amount effective, at dosages and for periods of time necessary, to achieve the desired therapeutic, diagnostic or prophylactic result.

An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

A “control subject” refers to a healthy subject who has not been diagnosed as having a neurodegenerative disorder (e.g., AD) and who does not suffer from any sign or symptom associated with a neurodegenerative disorder (e.g., AD). Control subjects may also include healthy subjects which have no familial history of a neurodegenerative disorder, such as AD.

The term “sample,” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue” or “cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines (e.g., neurons). Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method of the invention is being used to identify. A reference sample, reference cell, reference tissue, control sample, control cell, or control tissue may be obtained from a healthy part of the body of the same subject or patient in whom a disease or condition is being identified using a composition or method of the invention. A reference sample, reference cell, reference tissue, control sample, control cell, or control tissue may alternatively be obtained from a healthy part of the body of an individual who is not the subject or patient in whom a disease or condition is being identified using a composition or method of the invention. The gene expression level from a “reference sample”, “reference cell”, “reference tissue”, “control sample”, “control cell”, or “control tissue” may also be a predetermined as an average of levels obtained from a population that is not afflicted with a neurodegenerative disease or disorder, but in some instances, the reference level can be a mean or median level from a group of individuals including patients with a neurodegenerative disease or disorder.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” or “response” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e., reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

The term “gene signature” is used interchangeably with “gene expression signature” and refers to one or a combination of genes whose expression is indicative of a neurodegenerative disorder, e.g. AD, characterized by certain molecular, pathological, histological, and/or clinical features. In certain embodiments, the expression of one or more genes comprising the gene signature is elevated compared to that in control subjects.

The term “protein signature” is used interchangeably with “protein expression signature” and refers to one or a combination of proteins whose expression is indicative of neurodegenerative disorder, e.g. AD, characterized by certain molecular, pathological, histological, and/or clinical features. In certain embodiments, the expression of one or more proteins comprising the protein signature is elevated compared to that in control subjects.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is a minimum antibody fragment which contains a complete antigen-binding site. In one embodiment, a two-chain Fv species consists of a dimer of one heavy- and one light-chain variable domain in tight, non-covalent association. Collectively, the six CDRs of an Fv confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; U.S. Pat. No. 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6855-9855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 55-93 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “affinity matured” antibody is one with one or more alterations in one or more CDRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

Antibody “effector functions” refer to those biological activities attributable to the Fc region (a native-sequence Fc region or amino-acid-sequence-variant Fc region) of an antibody, and vary with the antibody isotype. Examples of antibody effector functions include but are not limited to: Clq binding and complement-dependent cytotoxicity (CDC); Fc-receptor binding; antibody-dependent cell-mediated cytotoxicity (ADCC); phagocytosis; down-regulation of cell-surface receptors (e.g. B-cell receptor); and B-cell activation.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural-killer (NK) cells, monocytes, cytotoxic T cells, and neutrophils. The effector cells may be isolated from a native source, e.g., from blood.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art.

The word “label” when used herein refers to a detectable compound or composition. The label is typically conjugated or fused directly or indirectly to a reagent, such as a nucleic acid probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

An “isolated” biological molecule, such as a nucleic acid, polypeptide, or antibody, is one which has been identified and separated and/or recovered from at least one component of its natural environment.

Reference to “about” a value or parameter herein includes (and describes) embodiments that are directed to that value or parameter per se. For example, description referring to “about X” includes description of “X.”

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic or diagnostic products or medicaments, that contain information about the indications, usage, dosage, administration, contraindications, other therapeutic or diagnostic products to be combined with the packaged product, and/or warnings concerning the use of such therapeutic or diagnostic products or medicaments and the like.

A “kit” is any manufacture (e.g., a package or container) comprising at least one reagent, e.g., a probe for specifically detecting a biomarker gene or protein of the invention. In certain embodiments, the manufacture is promoted, distributed, or sold as a unit for performing the methods of the present invention.

The expression “not responsive to,” as it relates to the reaction of subjects or patients to one or more of the medicaments that were previously administered to them, describes those subjects or patients who, upon administration of such medicament(s), did not exhibit any or adequate signs of treatment of the disorder for which they were being treated, or they exhibited a clinically unacceptably high degree of toxicity to the medicament(s), or they did not maintain the signs of treatment after first being administered such medicament(s), with the word treatment being used in this context as defined herein. The phrase “not responsive” includes a description of those subjects who are resistant and/or refractory to the previously administered medication(s), and includes the situations in which a subject or patient has progressed while receiving the medicament(s) that he or she is being given, and in which a subject or patient has progressed within 12 months (for example, within six months) after completing a regimen involving the medicament(s) to which he or she is no longer responsive. The non-responsiveness to one or more medicaments thus includes subjects who continue to have active disease following previous or current treatment therewith. For instance, a patient may have active disease activity after about one to three months of therapy with the medicament(s) to which they are non-responsive. Such responsiveness may be assessed by a clinician skilled in treating the disorder in question.

The “amount” or “level” of a biomarker as used in the methods of the present invention is a detectable level in a biological sample. These can be measured by methods known to one skilled in the art and also disclosed herein.

The terms “level of expression” or “expression level” in general are used interchangeably and generally refer to the amount of a polynucleotide or an amino acid product or protein in a biological sample. “Expression” generally refers to the process by which gene-encoded information is converted into the structures present and operating in the cell. Therefore, as used herein, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer, non-coding RNAs (ncRNA) and ribosomal RNAs (rRNA)).

### Biomarkers for Use in the Methods of the Invention

The biomarkers for use in the methods of the present invention include, for example the expression products (e.g. protein, mRNA, ncRNA, or other polynucleotide) of the genes tbx6 and dleu2.

Tbx6, also known as T-box transcription factor 6 or T-box protein 6, is a transcriptional regulator involved in developmental processes. See UnitProtKB/Swiss Prot Entry Number 095947, which is incorporated herein by reference in its entirety. Tbx6 is a member of the T-box gene family and has the chromosomal location of 16p12-q12 in humans. See Yi et al., Genomics 55:10-20 (1999), which is incorporated herein by reference in its entirety.

The T-box 6 protein is 436 amino acids in length and contains one T-box DNA binding domain. See UnitProtKB/Swiss Prot Entry Number 095947. Natural variants of Tbx6 are known to exist such as a GLY to SER substitution at amino acid 162, a SER to PHE substitution at amino acid 178 and a PRO to SER substitution at amino acid 179. Id. Several sequences for tbx6 have been deposited in GenBank and have the following accession numbers: AJ007989 (mRNA) and CAA07812.1 (translation); BCO26031 (mRNA) and AAH26031.1 (translation); AJ010279 (genomic DNA) and CAB37938.1 (translation) and are all incorporated herein by reference in their entireties.

Dleu2 encodes a long noncoding RNA (ncRNA) that is between 1.0-1.8 kb in length and is polyadenylated and spliced. See Klein et al., Cancer Cell 17: 28-40 (January 2010), incorporated herein by reference in its entirety. The ncRNA is also known as LEU2. UnitProtKB/Swiss Prot Entry Number 043262. The function of dleu2 is unknown, but other members of this class of ncRNAs have functions ranging from X chromosome inactivation or activation, imprinting, and transcriptional activation/regulation of gene expression. Klein et al., Cancer Cell 17: 28-40 (January 2010). Dleu2 is located at human chromosomal region 13q14 in a gene cluster with dleu1 and the micro RNAs miR-15a/16-1. Id. It has been shown that the dleu2/miR-15a/16-1 locus plays a role in the expansion of mature B cells. Id. Furthermore, the locus has a tumor-suppressor role in B cells and deletion of the locus in mice causes B cell chronic lymphocytic leukemia (CLL) associated phenotypes. Id.

A hypothetical protein of 55 amino acids could be encoded by the ncRNA. See UnitProtKB/Swiss Prot Entry Number 043262. Several sequences for delu2 have been deposited in GenBank and have the following accession numbers: Y15228 (mRNA) and CAA75516.1 (translation); CH471075 (genomic DNA) and EAX08851.1 (translation); BC017819 (mRNA) and AAH17819.1 (translation); BCO22282 (mRNA) and AAH22282.1 (translation); BCO30971 (mRNA) and AAH30971.1 (translation) and are all incorporated by reference in their entireties. Id.

Other neurodegenerative biomarkers known in the art may also be used in combination with tbx6 and/or dleu2 in the methods of the invention. Additional neurodegenerative biomarkers include, for example, amyloid-β (Aβ), amyloid precursor protein (APP), tau, presenilin 1 (PS1), presenilin 2 (PS2), apolipoprotein E (apoE), neuronal thread protein (NTP), α-antichymotripsin, β-secretase, CD59, C-reactive protein, Clq, 8-hydroxy-deoxyguanine, glutamine synthase, glial fibrillary acidic protein (GFAP), IL-6 receptor complex, kallikrein, melanotransferin, neurofiliment proteins, nitrotyrosine, oxysterols, sulphatides, synaptic markers, S100β and other neurodegenerative biomarkers mentioned in U.S. Published Application Nos. 2010/0255485; 2010/0167947; 2010/0159486; 2010/0124756; 2009/0239241; 2008/0261226; 2008/0220449; 2008/0026405; 2005/0244890; 2005/0221348; U.S. Pat. Nos. 4,728,605, 5,874,312, 6,027,896, 6,114,133, 6,130,048, 6,210,895, 6,358,681, 6,451,547, 6,461,831, 6,465,195, 6,475,161, and 6,495,335. Additional neurodegenerative biomarkers include those mentioned in Fahnestock et al., J. Neural. Transm. Suppl. 62:241-52 (2002); Masliah et al., Neurobiol. Aging 16(4):549-56 (1995); Power et al., Dement. Geriatr. Cogn. Disord. 12(2):167-70 (2001); and Burbach et al., J. Neurosci. 24(10):2421-30 (2004).

One of skill in the art will know how to construct probes for use in the methods of the present invention which target neurodegenerative biomarkers based on the information provided herein, as well as information in the art.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction, (Mullis et al., eds., 1994).

Primers, oligonucleotides and polynucleotides employed in the present invention can be generated using standard techniques known in the art.

The sample can be obtained by a variety of procedures known in the art including, but not limited to surgical excision, aspiration or biopsy. The tissue may be fresh or frozen. In one embodiment, the sample is fixed and embedded in paraffin or the like. The tissue sample may be fixed (i.e. preserved) by conventional methodology. One of ordinary skill in the art will appreciate that the choice of a fixative is determined by the purpose for which the sample is to be histologically stained or otherwise analyzed. One of ordinary skill in the art will also appreciate that the length of fixation depends upon the size of the tissue sample and the fixative used.

Detection of Gene Expression Levels

As discussed below, expression of biomarkers in a sample can be analyzed by a number of methodologies, many of which are known in the art and understood by the skilled artisan, including but not limited to, immunohistochemical and/or Western analysis, quantitative assays such as ELISA, ELIFA, in situ hybridization, immunoprecipitation, molecular binding assays, microarray analysis, fluorescence activated cell sorting (FACS) Northern analysis and or PCR analysis of RNAs such as mRNAs and ncRNAs, as well as any of the wide variety of assays that can be performed by gene and/or tissue array analysis. Typical protocols for evaluating the status of genes and gene products are found, for example, in Ausubel, et al., eds., 1995, Current Protocols in Molecular Biology, Units 2 (Northern Blotting), 4 (Southern Blotting), 15 (Immunoblotting), and 18 (PCR analysis).

Additional methods of detecting expression of biomarkers in a mammalian tissue or cell sample include contacting the sample with an antibody which binds the biomarker, reactive fragment thereof, or a recombinant protein containing an antigen binding region of a biomarker protein and then detecting the binding of the antibody, fragment thereof or recombinant protein, in the sample.

In particular embodiments of the invention, the expression of biomarkers in a sample is examined using immunohistochemistry and staining protocols. Immunohistochemical staining of tissue sections has been shown to be a reliable method of assessing or detecting presence of proteins in a sample. Immunohistochemistry (“IHC”) techniques utilize an antibody to probe and visualize cellular antigens in situ, generally by chromogenic or fluorescent methods.

For sample preparation, a tissue or cell sample from a mammal (e.g., a human brain tissue sample) may be used. Examples of samples include, but are not limited to, tissue biopsy, brain tissue biopsy, blood, lung aspirate, sputum, lymph fluid, etc. Genes or gene products can be detected from disease tissue or from other body samples, for example, brain tissue (biopsy), cerebrospinal fluid, blood, including whole blood, plasma or serum, urine, saliva, tears, etc. In certain instances, individual cells or cell types may be isolated such as, but not limited, to neurons. The sample can be obtained by a variety of procedures known in the art including, but not limited to surgical excision, aspiration or biopsy. The tissue may be fresh or frozen. In one embodiment, the sample is fixed and embedded in paraffin or the like. A biological sample from a subject can be obtained by methods well known in the art. Tissue biopsy is often used to obtain a representative piece of diseased tissue. Alternatively, cells can be obtained indirectly in the form of tissues/fluids that are known or thought to contain the disease cells of interest. For sample preparation, a tissue or cell sample from a mammal (typically a human patient) may be used.

The tissue sample may be fixed (i.e. preserved) by conventional methodology (See e.g., Manual of Histological Staining Method of the Armed Forces Institute of Pathology, 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a sample.

Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., Manual of Histological Staining Method of the Armed Forces Institute of Pathology, supra). Examples of paraffin that may be used include, but are not limited to, Paraplast, Broloid, and Tissuemay. Once the tissue sample is embedded, the sample may be sectioned by a microtome or the like (See e.g., Manual of Histological Staining Method of the Armed Forces Institute of Pathology, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., Manual of Histological Staining Method of the Armed Forces Institute of Pathology, supra). Alternatively, commercially available deparaffinizing non-organic agents such as Hemo-De7 (CMS, Houston, Tex.) may be used.

Optionally, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target antigen (e.g., a biomarker) is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.

(b) Colloidal gold particles.

(c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example, and fluorescence can be quantified using a fluorimeter.

(d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., “Methods for the Preparation of Enzyme-Antibody Conjugates for use in Enzyme Immunoassay”, in Methods in Enzym. (ed. J. Langone & H. Van Vunakis), Academic press, New York, 73:147-166 (1981).

Examples of enzyme-substrate combinations include, for example: (i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired. For example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Optionally, the antibodies employed in the IHC analysis to detect expression of a biomarker are antibodies generated to bind primarily to the biomarker of interest. Optionally, the anti-biomarker antibody is a monoclonal antibody. Anti-biomarker antibodies are readily available in the art, including from various commercial sources, and can also be generated using routine skills known in the art.

Specimens thus prepared may be mounted and coverslipped. Slide evaluation is then determined, e.g. using a microscope, and staining intensity criteria, routinely used in the art, may be employed. As one example, staining intensity criteria may be evaluated as follows:

TABLE 1

| Staining Pattern | Score |
|---|---|
| No staining is observed in cells. | 0 |
| Faint/barely perceptible staining is detected in | 1+ |
| more than 10% of the cells. | |
| Weak to moderate staining is observed in | 2+ |
| more than 10% of the cells. | |
| Moderate to strong staining is observed in | 3+ |
| more than 10% of the cells. | |

In alternative methods, the sample may be contacted with an antibody specific for a biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labelled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C., such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the enzyme immunoassay (EIA), the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

It is contemplated that the above described techniques may also be employed to detect expression of a biomarker such as tbx6 or dleu2.

Methods of the invention further include protocols which examine the presence and/or expression of ncRNAs and/or mRNAs, such as tbx6 mRNA or dleu2 ncRNA, in a tissue or cell sample. Methods for the evaluation of ncRNAs and/or mRNAs in cells are well known and include, for example, hybridization assays using complementary DNA probes (such as in situ hybridization using labeled biomarker riboprobes, Northern blot and related techniques) and various nucleic acid amplification assays (such as RT-PCR using complementary primers specific for biomarkers, and other amplification type detection methods, such as, for example, branched DNA, SISBA, TMA and the like).

Tissue or cell samples from mammals can be conveniently assayed for, e.g., biomarker mRNAs and/or ncRNAs using Northern, dot blot or PCR analysis. For example, RT-PCR assays such as quantitative PCR assays are well known in the art. In an illustrative embodiment of the invention, a method for detecting a biomarker mRNA and/or ncRNA in a biological sample comprises producing cDNA from the sample by reverse transcription using at least one primer; amplifying the cDNA so produced using a biomarker polynucleotide as sense and antisense primers to amplify biomarker cDNAs therein; and detecting the presence of the amplified biomarker cDNA. In addition, such methods can include one or more steps that allow one to determine the levels of biomarker mRNA and/or ncRNA in a biological sample (e.g. by simultaneously examining the levels a comparative control mRNA sequence of a “housekeeping” gene such as an actin family member). Optionally, the sequence of the amplified biomarker cDNA can be determined.

Biomarker primers and primer pairs, which allow the specific amplification of the polynucleotides for use in the methods of the invention or of any specific parts thereof, and probes that selectively or specifically hybridize to nucleic acid molecules for use in the methods of the invention or to any part thereof. Probes may be labeled with a detectable marker, such as, for example, a radioisotope, fluorescent compound, bioluminescent compound, a chemiluminescent compound, metal chelator or enzyme. Such probes and primers can be used to detect the presence of biomarker polynucleotides in a sample and as a means for detecting a cell expressing biomarker proteins. As will be understood by the skilled artisan, a great many different primers and probes may be prepared based on the sequences provided herein and used effectively to amplify, clone and/or determine the presence and/or levels of biomarker mRNAs and/or ncRNAs.

Optional methods of the invention include protocols which examine or detect mRNAs and/or ncRNAs, such as tbx6 and dleu2 mRNAs and ncRNAs, in a tissue or cell sample by microarray technologies. Using nucleic acid microarrays, test and control mRNA and/or ncRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment. (see, e.g., WO 01/75166 published Oct. 11, 2001; (see, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). The Affymetrix GeneChip™ system is an example of one commercially available microarray system which comprises arrays fabricated by direct synthesis of oligonucleotides on a glass surface.

The expression of a selected biomarker may also be assessed by examining gene deletion or gene amplification. Gene deletion or amplification may be measured by any one of a wide variety of protocols known in the art, for example, by conventional Southern blotting, Northern blotting to quantitate the transcription of mRNA and/or ncRNA (Thomas, Proc. Natl. Acad. Sci. USA, 77:5201-5205 (1980)), dot blotting (DNA analysis), or in situ hybridization (e.g., FISH), using an appropriately labeled probe, cytogenetic methods or comparative genomic hybridization (CGH) using an appropriately labeled probe. By way of example, these methods may be employed to detect deletion or amplification of biomarker genes.

In Vivo Detection

In one aspect, a probe for use in the methods of the invention is administered to a patient in an amount or dosage suitable for in vivo imaging. Generally, the amount of probe needed for use in the methods of the invention will vary depending on patient considerations. Such considerations include, for example, age, protocol, condition, sex, extent of disease, weight, contraindications, concomitant therapies and the like. An exemplary amount of probe for imaging can be determined, adjusted or modified by a physician skilled in the art, based on these considerations. For example, a unit dosage for a patient comprising a probe for use in the methods of the invention can vary from 1×10⁻¹⁵ g/kg to 10 g/kg, preferably, 1×10⁻¹⁵ g/kg to 1.0 g/kg. Moreover, a unit dosage comprising a probe for use in the methods of the present invention can also be from 1 μCi/kg to 10 mCi/kg and, preferably, 0.1 mCi/kg. Dosage of a probe for use in the methods of the invention can also vary from 0.001 μg/kg to 10 μg/kg or, preferably, from 0.01 μg/kg to 1.01μ/kg. An effective amount of probe for use in the methods of the invention administered to a subject as ocular drops can also be adjusted or modified by one skilled in the art.

Administration of probe to a subject in the methods of the invention may be local or systemic and accomplished intravenously, intraarterially, intrathecally (via the spinal fluid), intraocularly or the like. Administration may also be intradermal or intracavitary.

In one aspect, after a sufficient time has elapsed for a probe for use in the methods of the invention to bind with a target, the area of the subject under investigation is examined by routine imaging techniques or modalities such as magnetic resonance spectroscopy (MRS), magnetic resonance spectroscopy imaging (MRI), positron emission tomography (PET), single-photon emission computed tomography (SPECT), planar scintillation imaging or combinations thereof as well as any emerging imaging modalities or others described herein. The exact protocol will necessarily vary depending upon factors specific to the patient and depending upon the method of administration and type of probe or detectable marker used, although the determination of specific procedures would be routine to the skilled artisan.

The probes for use in the methods of the invention can also be administered in the form of injectable compositions, but may also be formulated into well known drug delivery systems such as, for example, oral, rectal, parenteral (intravenous, intramuscular, or subcutaneous), intracisternal, intravaginal, intraperitoneal, local (powders, ointments or drops) or as a buccal or nasal spray as well as ocular drops. A typical composition for administration can comprise a pharmaceutically acceptable carrier for the probe for use in the methods of the invention. A pharmaceutically acceptable carrier includes such carriers as, for example, aqueous solutions, non-toxic excipients including salts, preservatives, buffers and the like, which are described in Remington's Pharmaceutical Sciences, 15th Ed. Easton: Mack Publishing Co., pp. 1405-1412 and 1461-1487 (1975) and The National Formulary XIV., 14th Ed. Washington: American Pharmaceutical Association (1975).

In one aspect, probes for use in the methods of the invention are those that, in addition to binding (for example, preferentially or specifically) biomarkers described herein in vivo, are capable of crossing the blood brain barrier (BBB), and are non-toxic at appropriate dosage levels and have a satisfactory duration of effect.

Several art-known approaches exist for transporting molecules across the blood-brain barrier, including, but not limited to, physical methods, lipid-based methods, stem cell-based methods, and receptor and channel-based methods.

Physical methods of transporting a probe across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002)), interstitial infusion/convection-enhanced delivery (see, e.g., Bobo et al., Proc. Natl. Acad. Sci. USA 91: 2076-2080 (1994)), and implanting a delivery device in the brain (see, e.g., Gill et al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989)), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the probe (see, e.g., U.S. Patent Publication No. 2003/0083299).

Lipid-based methods of transporting a probe across the blood-brain barrier include, but are not limited to, encapsulating the probe in liposomes that are coupled to antibody binding fragments that bind to receptors on the vascular endothelium of the blood-brain barrier (see, e.g., U.S. Patent Application Publication No. 2002/0025313), and coating the probe in low-density lipoprotein particles (see, e.g., U.S. Patent Application Publication No. 2004/0204354) or apolipoprotein E (see, e.g., U.S. Patent Application Publication No. 2004/0131692).

Stem-cell based methods of transporting a probe across the blood-brain barrier entail genetically engineering neural progenitor cells (NPCs) to express the probe of interest and then implanting the stem cells into the brain of the individual to be treated. See Behrstock et al. Gene Ther. 15 Dec. 2005 advanced online publication (reporting that NPCs genetically engineered to express the neurotrophic factor GDNF reduced symptoms of Parkinson disease when implanted into the brains of rodent and primate models).

Receptor and channel-based methods of transporting a probe across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the antibodies (see, e.g., U.S. Pat. No. 5,004,697).

Additionally, probes for use in the methods of the present invention may be conjugated or associated with a brain-targeting peptide. A “brain-targeting peptide” as used herein is a protein (e.g., a ligand or a peptidomimetic antibody) which is normally transported (e.g., via carrier-mediated transport or receptor-mediated transport) through the BBB. Non-limiting examples of such brain-targeting peptides include insulin or transferrin. Additional brain targeting peptides include receptor specific peptidomimetic antibodies, or fragments thereof, which bind to transport receptors such as insulin receptor, transferrin receptor, leptin receptor, GLUT1 glucose transporter, MCT1 lactate transporter, LAT1 large neutral amino acid transporter, and CNT2 adenosine transporter to facilitate transport across the BBB.

In certain aspects, the probes for use in the methods of the present invention are peptide nucleic acids (PNA), in which a polynucleotide is conjugate to or associated with a brain targeting polypeptide.

The location of biomarker for use in the methods of the invention may be taken into consideration in preparation and administration of the probe. When the binding target is an intracellular molecule, certain embodiments of the invention provide for the probe to be introduced into the cell where the binding target is located. In one embodiment, a probe of the invention can be expressed intracellularly, e.g. an intrabody. The term “intrabody,” as used herein, refers to an antibody or antigen-binding portion thereof that is expressed intracellularly and that is capable of selectively binding to a target molecule, as described, e.g., in Marasco, Gene Therapy 4: 11-15 (1997); Kontermann, Methods 34: 163-170 (2004); U.S. Pat. Nos. 6,004,940 and 6,329,173; U.S. Patent Application Publication No. 2003/0104402, and PCT Publication No. WO2003/077945. See also, for example, WO96/07321 published Mar. 14, 1996, concerning the use of gene therapy to generate intracellular antibodies.

Intracellular expression of a probe may be effected by introducing a nucleic acid encoding the desired probe into a target cell. One or more nucleic acids encoding all or a portion of the probe can be delivered to a target cell, such that one or more probes are expressed which are capable of binding to an intracellular target biomarker. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest.

In certain embodiments, nucleic acid (optionally contained in a vector) may be introduced into a patient's cells by in vivo a methods. In one example of in vivo delivery, nucleic acid is injected directly into the patient, e.g., at the site if the neurodegenerative disease or disorder. In a further example of in vivo delivery, nucleic acid is introduced into a cell using transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of certain gene marking and gene therapy protocols, see Anderson et al., Science 256:808-813 (1992), and WO 93/25673 and the references cited therein.

The invention employs probes which, in conjunction with noninvasive neuroimaging techniques or modalities such as MRS, MRI, PET or SPECT, are used to quantify gene expression in vivo. The methods of the invention also involve imaging a patient to establish a baseline of biomarker gene expression. An exemplary method of the invention comprises at least one imaging session of a patient following administration of a therapy. In one aspect, a method of the invention may involve imaging, a patient before and after treatment with at least one therapeutic agent. In vivo imaging may also be performed at any time during the treatment.

Probes for use in the methods of the invention can be labeled (i.e., marked or tagged) for imaging or detection. Any suitable label (radiolabel or tag) may be used for detection of a biomarker probe.

Exemplary techniques for detection of a biomarker probe include scintigraphy, radioscintigraphy, magnetic resonance imaging (MRI), chemilumensence, near infrared luminescence, fluorescence, SPECT, computed tomography (CT scan), positron emission tomography (PET) or combinations thereof. Detection and related techniques are understood by those of ordinary skill in the art.

For purposes of in vivo imaging, the type of detection instrument is a factor in selecting a given detectable marker. For example, radioactive isotopes and ¹⁸F or ¹²³I are suitable for in vivo imaging in the methods of the invention. The type of instrument used will also guide the selection of a radionuclide or stable isotope. In one aspect, the radionuclide chosen must have a type of decay detectable by a given type of instrument. Moreover, other considerations such as the half-life of the radionuclide are taken into account when selecting a detectable marker for in vivo imaging. Imagining techniques are known in the art and one of ordinary skill in the art will be able to choose an appropriate detectable marker for use in the methods of the invention.

The half-life of a detectable marker should be long enough so that the marker is still detectable at the time of maximum uptake by the target, but short enough so that the subject does not sustain deleterious radiation. The probes for use in the methods of the invention can be detected using gamma imaging in which emitted gamma irradiation of the appropriate wavelength is detected. Conventional methods of gamma imaging include, but are not limited to, SPECT and PET. Preferably, for SPECT detection, the chosen detectable marker will lack a particulate emission, but will produce a large number of photons in a 140-300 keV range. For PET detection, the detectable marker will be a positron-emitting radionuclide such as ¹⁸F, which will annihilate to form two 511 keV gamma rays that can then be detected by a PET camera.

In one aspect, probes for use in the methods of the invention, which are useful for in vivo imaging are administered to a subject. The probes are used in conjunction with non-invasive neuroimaging techniques such as MRS, MRI, PET, SPECT and/or combinations thereof. Probes for use in the methods of the invention may be labeled with ¹⁹F or ¹³C to yield a probe for MRS/MRI using general organic chemistry techniques known to the art. March, J., Advanced Organic Chemistry: I Reactions, Mechanisms, and Structure (3rd Ed., 1985); Morrison and Boyd, Organic Chemistry (6th Ed., 1992). The probes for use in the methods of the invention may also be radiolabeled with ¹⁸F, ¹¹C, ⁷⁵Br or ⁷⁶Br for PET by techniques well known in the art and described by Fowler, J. and Wolf, A. in Positron Emission Tomography and Autoradiography (Phelps, M., Mazziota, J., and Schelbert, H., eds.) pp. 391-450 (Raven Press, NY 1986). The probes for use in the methods of the invention also may be radiolabeled with ¹²³I for SPECT by any of several techniques known to the art. Kulkarni, Int. J. Rad. Appl. & Inst., (Part B) 18: 647 (1991).

A label, detectable label, radiolabel, tag, marker, detectable marker, tracer, radiotracer or equivalent term as generally understood by those of ordinary skill in the art can represent any substituent (group, moiety, position) suitable for imaging and/or assaying (for example, identifying, diagnosing, evaluating, detecting and/or quantitating). For example, a probe for use in the methods of the invention can comprise labels, radiolabels, tags, markers, detectable markers, tracers, radiotracers or equivalent terms suitable for in vivo or in vitro detection via radioscintigraphy, magnetic resonance imaging (MRI), assays, chemilumensence, near infrared luminescence, fluorescence, spectroscopy, gamma imaging, magnetic resonance imaging, magnetic resonance spectroscopy, fluorescence spectroscopy, SPECT, computed tomography (CT scan), positron emission tomography (PET). Suitable labels, radiolabels, tags, markers, detectable markers, tracers, radiotracers or equivalent terms are known by those skilled in the art and can include, for example, radioisotopes, radionuclides, isotopes, fluorescent groups, biotin (in conjunction with streptavidin complexation) or photoaffinity groups. A label, detectable label, radiolabel, tag, marker, detectable marker, tracer, radiotracer of a probe for use in the methods of the invention can comprise ¹³¹I, ¹²⁴I, ¹²⁵I, ³H, ¹²³I, ¹⁸F, ¹⁹F, ¹¹C, ⁷⁵Br, ¹³C, ¹³N, ¹⁵O, ⁷⁶Br, “Photoaffinity group” or “photoaffinity labeled” can refer to a substituent on a probe for use in the methods of the invention, which can be activated by photolysis at an appropriate wavelength to undergo a cross-linking photochemical reaction with a macromolecule associated therewith. An example of a photoaffinity group is a benzophenone substituent.

Suitable radioisotopes are known to those skilled in the art and include, for example, isotopes of halogens (such as chlorine, fluorine, bromine and iodine) and metals including technetium and indium. Exemplary labels, radiolabels, tags, markers, detectable markers, tracers, radiotracers can also include ³H, ¹¹C, ¹⁴C, ¹⁸F, ³²F, ³⁵S, ¹²³I, ¹²⁵I, ¹³¹I, ¹²⁴I, ¹⁹F, ⁷⁵Br, ¹³C, ¹³N, ¹⁵O, ⁷⁶Br. The probes of use in the methods of the invention may be labeled (radiolabeled, tagged, marked, detectably marked, traced or radiotraced) either directly (that is, by incorporating the label directly into a compound of the invention) or indirectly (that is, by incorporating the label into a compound of the invention through a chelating agent, where the chelating agent has been incorporated into the compound). Furthermore, a label for a probe can be included as an additional substituent (group, moiety, position) to a compound of the invention or as an alternative substituent for any substituents that are present. A label, detectable label, radiolabel, tag, marker, detectable marker, tracer or radiotracer may appear at any substituent (group, moiety, position) on a probe for use in the methods of the invention.

In one aspect, labeling can be isotopic or nonisotopic. With isotopic labeling, one substituent (group, moiety, position) already present in a probe for use in the methods of the invention can be substituted with (exchanged for) a radioisotope or isotope. With nonisotopic labeling, a radioisotope or isotope can be added to a probe for use in the methods of the invention without substituting with (exchanging for) an already existing group.

In addition, the probes for use in the methods of the invention may be labeled with any suitable radioactive iodine isotope such as, but not limited to, ¹³¹I, ¹²⁵I or ¹²³I by iodination of a diazotized amino derivative directly via a diazonium iodide (Greenbaum, F., Am. J. Pharm., 108: 17 (1936)), by conversion of the unstable diazotized amine to the stable triazene or by conversion of a non-radioactive halogenated precursor to a stable tri-alkyl tin derivative, which then can be converted to an iodo compound by several methods well known to the art. Satyamurthy and Barrio, J. Org. Chem., 48: 4394 (1983), Goodman et al., J. Org. Chem., 49: 2322 (1984), Mathis et al., J. Labell. Comp. and Radiopharm., 1994: 905; Chumpradit et al., J. Med. Chem., 34: 877 (1991); Zhuang et al., J. Med. Chem., 37: 1406 (1994); Chumpradit et al., J. Med. Chem., 37: 4245 (1994). For example, a stable form or derivative of a compound of the invention can be reacted with a halogenating agent containing ¹³¹I, ¹²⁵I, ¹²³I, ⁷⁵Br, ⁷⁶Br or ¹⁸F.

The probes for use in the methods of the invention also may be radiolabeled with known metal detectable markers such as Technetium-99m (⁹⁹mTc). Modification of the substituents of a probe for use in the methods of the invention in order to introduce ligands that bind such metal ions can be effected without undue experimentation by one of ordinary skill in the art. Preparing probes comprising a detectable marker such as ⁹⁹mTc is well known in the art. Zhuang et al., Nuclear Medicine & Biology, 26(2): 217 (1999); Oya et al., Nuclear Medicine & Biology, 25(2): 135 (1998); Hom et al., Nuclear Medicine & Biology, 24(6): 485 (1997).

In one aspect, a method of the invention may use probes labeled with isotopes detectable by nuclear magnetic resonance (NMR) spectroscopy for purposes of in vivo imaging and spectroscopy. Elements particularly useful in magnetic resonance spectroscopy include ¹H, ¹⁹F and ¹³C. Suitable detectable markers for preparing a probe for use in the methods of the invention also include beta-emitters, gamma-emitters, positron-emitters and x-ray emitters. Moreover, exemplary detectable markers include ¹³¹I, ¹²³I, ¹²⁴I, ¹²⁵I, ³H, ¹²³I, ¹⁸F, ¹⁹F, ¹³C, ¹⁴C, ⁷⁵Br, ¹¹C, ¹³N, ¹⁵O and ⁷⁶Br. Any conventional method or detectable markers for visualizing probes for use in the methods of the invention can be used and will be appreciated by those of ordinary skill in the art.

Biomarker Expression Levels in Reference Sample

The expression level from reference samples used for comparison with the measured levels for at least one of the genes tbx6 and dleu2, depends on the method of the invention being practiced. For neurodegenerative disease or disorder diagnosis methods, the expression level from a “reference sample” is typically a predetermined reference level, such as an average of levels obtained from a population that is not afflicted with a neurodegenerative disease or disorder, but in some instances, the reference level can be a mean or median level from a group of individuals including patients with a neurodegenerative disease or disorder. In some instances, the predetermined reference level is derived from (e.g., is the mean or median of) levels obtained from an age-matched population.

For neurodegenerative disease or disorder monitoring methods (e.g., methods of diagnosing or aiding in the diagnosis of neurodegenerative disease or disorder progression in an patient with a neurodegenerative disease or disorder), the reference level may be a predetermined level, such as an average of levels obtained from a population that is not afflicted with a neurodegenerative disease or disorder, a population that has been diagnosed with a neurodegenerative disease or disorder, and, in some instances, the reference level can be a mean or median level from a group of individuals including patients with a neurodegenerative disease or disorder. Alternately, the reference level may be a historical reference level for the particular patient (e.g., a tbx6 level that was obtained from a sample derived from the same individual, but at an earlier point in time). In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

However, it will be appreciated by one of skill in the art that the level of expression of a biomarker in a reference sample may be determined by any method described herein as well.

Comparing Levels of tbx6 and/or dleu2

The process of comparing a measured value and a reference value can be carried out in any convenient manner appropriate to the type of measured value and reference value for the biomarker at issue. Measuring or determining the expression level of tbx6 and/or dleu2 can be performed using quantitative or qualitative measurement techniques, and the mode of comparing a measured value and a reference value can vary depending on the measurement technology employed. For example, when a qualitative colorimetric assay is used to measure gene expression levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). Measured or determined values used in the methods of the can also be quantitative values and depend on the method of detection used.

### Kits

For use in the applications described or suggested herein, kits or articles of manufacture are also provided. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising one or more genes of a gene expression signature. Where the kit utilizes nucleic acid hybridization to detect the target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label.

Kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above. Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

### EXAMPLES

The following are examples of the methods and compositions of the invention. It is understood that various other embodiments may be practiced, given the general description provided above.

### Example 1

### Characterization of Inhibitors with Respect to Localized Degeneration

Axon degeneration is a hallmark of both pruning during nervous system development and neurodegenerative disease. The molecular mechanisms regulating this active process are just beginning to be understood. In order to identify additional pathways regulating axon degeneration, an unbiased small molecule screen was conducted to identify modulators of various pathways that block axon degeneration following nerve growth factor withdrawal (NGF).

A number of kinases were identified in the screen as mediators of axon degeneration and further mechanistic studies, as described below, localized the function of distinct kinases to either the axonal or cell body compartments.

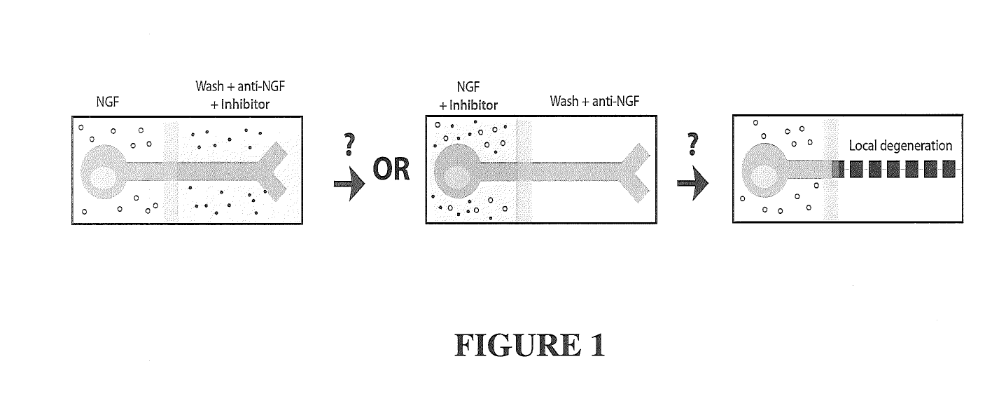

Campenot chambers were used to perform the following experiments which allow for the separation of somal and axonal environments, and permit the induction of localized degeneration (see FIG. 1 and, e.g., Zweifel et al., Nat. Rev. Neurosci. 6(8):615-625, 2005). In such chambers, axon degeneration is localized and proceeds without apoptosis.

Materials and Methods

Teflon dividers (Tyler Research) were cleaned by washing in water and wiping them clean of any residual grease. Dividers were then soaked in Nochromix (Godax Laboratories)/sulfuric acid overnight, rinsed five times in distilled and autoclaved water (SQ water), boiled for 30 minutes, and then air-dried before use.

Mouse laminin (5 μg/ml in sterile filtered water; Invitrogen) was added to PDL coated 35 mm dishes (BD Biosciences) and they were incubated for 1 hour at 37° C., followed by two rinses in SQ water. The dishes were vacuum-dried and then air-dried in a laminar flow hood for 15 minutes. Prepared dishes were then scored with a pin rake (Tyler Research). Fifty microliters of NBM+MC solution containing NGF was applied across the resulting score tracks. The NBM+MC solution was made as follows: 1750 mg of methylcellulose was combined with 480 ml of Neurobasal (Invitrogen), to which was added 4.5 ml penicillin/streptomycin, 7.5 ml L-glutamine, and 10 ml B-27 serum-free supplement (Invitrogen). The solution was mixed for one hour at room temperature, overnight at 4° C., and one further hour at room temperature. The solution was then filter sterilized, and 50 ng/ml NGF (Roche) was added prior to use. High vacuum grease (VWR) was added to each Teflon divider under a dissection scope. The laminin coated PDL dishes were inverted and dropped onto the Teflon divider, with additional pressure added by use of a toothpick in the non-track-containing regions. Dishes were incubated for 1 hour at 37° C. Five hundred microliters of NBM+MC (50 ng/ml NGF) solution was added to each of the side compartments, and a grease barrier was added in front of the center cell slot.

Free E13.5 spinal cords were dissected from mouse embryos and placed into NBM+MC (25 ng/ml NGF) solution. DRGs were detached from the spinal cord with a tungsten needle. An NBM+MC-lubricated P200 pipette was used to move DRGs into a 1.5 ml tube. DRGs were pelleted with a tabletop centrifuge for 30 seconds. The supernatant was discarded and 0.05% Trypsin/EDTA (cold) was added. The pellet was resolubilized with a pipette and incubated at 37° C. for 15 minutes with constant agitation (650 RPM). The sample was again centrifuged and the supernatant discarded. The pellet was resuspended in warm NBM+MC (50 ng/ml NGF) solution and triturated with a flamed glass pipette 20 times, followed by trituration with a fire-bored glass pipette another 20 times. The samples were again centrifuged and the resulting pellets were resuspended in 0.5 ml NBM+MC (50 ng/ml NGF) solution. The cells were diluted to a final concentration of 2.5×10⁶ cells/ml. The cell suspensions were loaded into a 1 ml syringe with a 22 gauge needle. The center slot of the Campenot divider was filled using the syringe (to a volume of at least 50 μl). The Campenot chamber was incubated overnight at 37° C. 2.5 ml NGF+MC (50 ng/ml NGF) solution was added to the center compartment and the grease gate was removed. The outer medium (cell body compartment) was replaced after three days with 2.5 ml NBM+MC medium (with 25 ng/ml NGF).

After five days in culture, the axonal compartment was washed three times with warmed NBM+MC (no NGF) solution. After the third wash, 500 μl NBM+MC (no NGF) solution was added to the axon compartment in combination with either 0.5% DMSO or an inhibitor. The cell body compartment was replaced with 2.5 ml NBM+MC medium (with 25 ng/ml NGF) containing either 0.5% DMSO or inhibitor. Fifty μg/ml anti-NGF antibody was added to the axonal compartment. Another axon compartment was maintained in NGF as a control.

After 28 hours of NGF deprivation for axons, and 25 hours of NGF deprivation for cell bodies, 8% PFA/30% sucrose solution was added directly to the culture medium at a 1:1 dilution and incubated for 30 minutes. The Teflon divider was removed after the first 15 minutes of addition. The system was washed once with 2.5 ml PBS prior to immunostaining Neurons were blocked in 5% BSA/0.2% triton in PBS for 30 minutes. The primary antibody Tuj1 (Covance) was added to a final dilution of 1:1000 in PBS containing 2% BSA and incubated overnight at 4° C. The dish was washed once with PBS. The secondary antibody (Alexa 488 goat anti-mouse antibody (Invitrogen)) was added at a final dilution of 1:200 in 2% BSA in PBS and incubated for one hour at room temperature. The dish was washed twice with PBS, and a 22×22 mm coverslip (VWR) was added with 350 μl of fluoromount G (Electron Microscopy Sciences). The neurons were visualized by use of a fluorescence microscope.

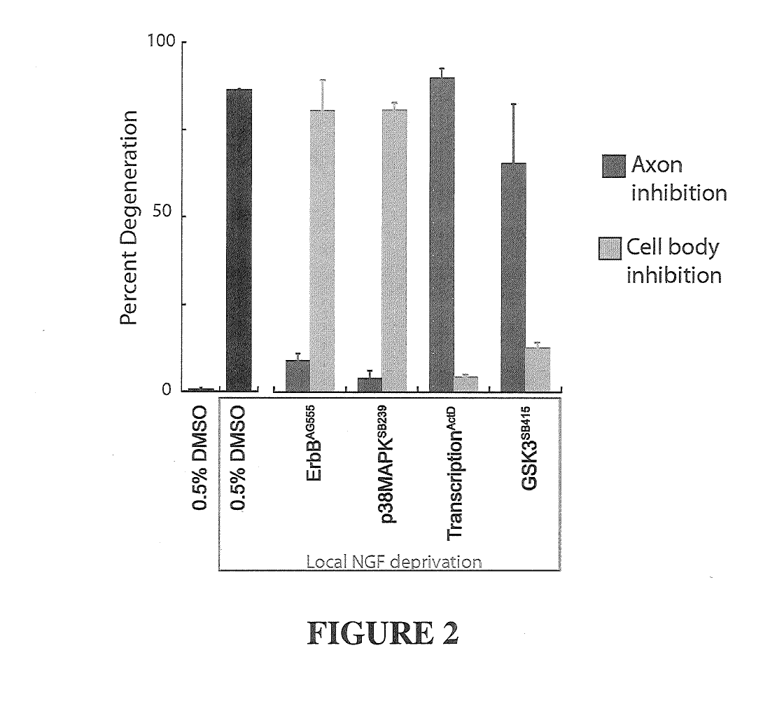

When axons were exposed to epidermal growth factor receptor (EGFR) kinase inhibitor AG555 (ErbB^{AG5555}) (EMB Biosciences) or the p38 MAP kinase inhibitor SB239 (p38MAPKSB²³⁹) (EMB Biosciences), axons deprived of NGF exhibited less degeneration. In contrast, AG555 and SB239 treatment in the cell body compartment, failed to prevent degeneration. When the cell body was treated with the transcription inhibitor Act D (Transcription^{ActD}) (Sigma) or the glycogen synthase kinase-3 (GSK-3) inhibitor SB415 (SK3^{SB415}) (Sigma) axon degradation due to NGF deprivation was reduced in the axon. However, the same inhibitors did not prevent axonal degradation due to NGF deprivation when applied directly to the axon. These results suggest that signaling in local axon degeneration is not limited to the axon segment being lost; some inhibitors are most effective when applied to the cell body, and others to the axon. Quantification of these results is shown in FIG. 2.

### Example 2

### Microarray Analysis to Identify GSK-3 Regulated Genes Involved in Axon Degeneration

Based on the experiments described above, glycogen synthase kinase-3 (GSK-3) was identified as being a regulator of a genetic axon degeneration program acting specifically in the cell body to regulate distal axon degeneration. To identify GSK3 regulated axon degeneration genes, time course microarray analysis was performed on neurons selectively undergoing neuron loss, with or without GSK-3 inhibition.

Briefly, neurons were isolated and cultured in Campenot chambers as previously described. Campenot chambers were set up with 50 μM 5-fluoro-2′-deoxyuridine/uridine (both Sigma) added to the culture medium to reduce contamination by non-neuronal cells. On day 5, both axon compartments were washed three times with NGF-free medium and on the fourth wash, replaced with medium containing NGF (control) or NGF antibodies (50 μg/ml) (911, Genetech). The outer compartment was replaced with medium containing 30 μM of the GSK3 inhibitor ARA (EMD Biosciences) or 0.3% DMSO. RNA was extracted from neurons with or without GSK3 ARA treatment which had been cultured with NGF or deprived of NGF for 6 and 12 hours. RNA was prepared with Trizol® (Invitrogen) followed by the RNeasy® Micro Kit processing with DNAse I treatment (Qiagen) as described in Appendix C of the RNeasy® Micro Kit manual. For each condition, five Agilent Whole Mouse Genome microarray chips were used with single amplification.

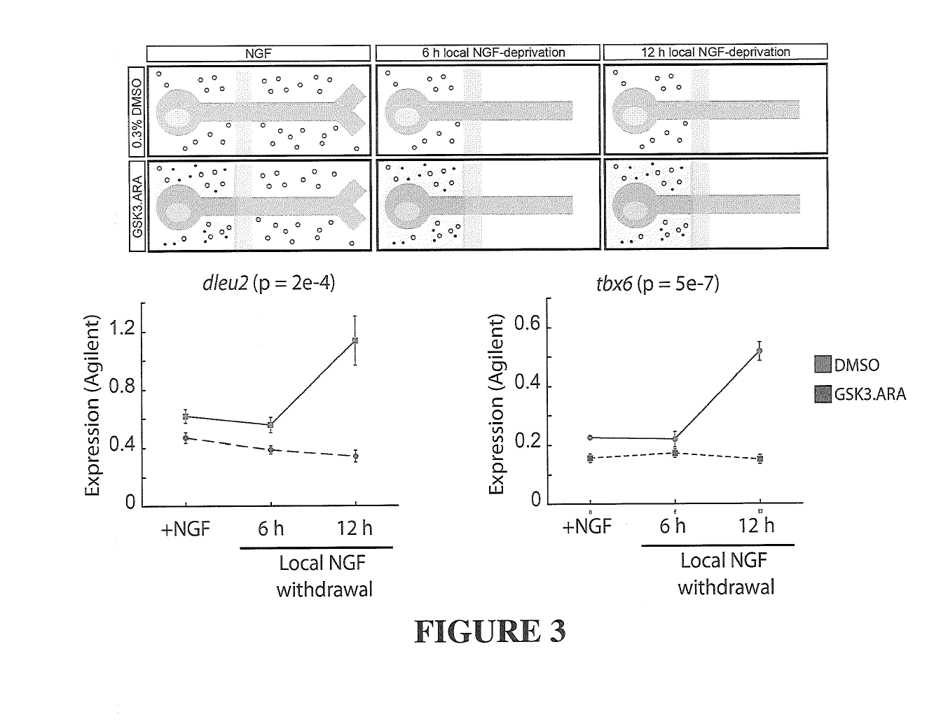

Two genes, tbx6 and dleu2 were identified as being overexpressed in neurons undergoing axon degeneration in the microarray analysis. Tbx6 encodes a transcription factor and dleu2 encodes a long noncoding RNA, as described previously. As can be seen in FIG. 3, dleu2 and tbx6 are both overexpressed in neurons after 12 hours of NGF deprivation. However, the overexpression of these genes is not observed at any time point with the addition of the GSK3 ARA inhibitor.

### Example 3

### Tbx6 and Dleu2 Knockdown Decreases Axonal Degeneration

In order to further assess the role of dleu2 and tbx6 in axonal degeneration, knockdown experiments were performed in which siRNA was used to reduce expression of dleu2 and tbx6 in neurons undergoing NGF deprivation.

Briefly, E13.5 mouse DRGs were dissociated after trypsin digestion. Three different siRNAs for dleu2 and tbx6 were tested individually. The following siRNAs were used:

sidleu2.1:
Sense
(SEQ ID NO: 1)
(5′-GAUAGGCGAUUAAGGUUUATT-3′)
Antisense
(SEQ ID NO: 2)
(5′-UUCAGCUGUGUGAUCCUAGGG-3′)
sidleu2.2:
Sense
(SEQ ID NO: 3)
(5′-CGGGAAUCAAACAAGUCUATT-3′)
Antisense
(SEQ ID NO: 4)
(5′-UAGACUUGUUUGAUUCCCGTT-3′)
sidleu2.3:
Sense
(SEQ ID NO: 5)
(5′-GAAACACGAUACUUCUUGATT-3′)
Antisense
(SEQ ID NO: 6)
(5′-UCAAGAAGUAUCGUGUUUCTG-3′)
sitbx6.1:
Sense
(SEQ ID NO: 7)
(5′-GAAGAAACUACAACAUGUATT-3′)
Antisense
(SEQ ID NO: 8)
(5′-UACAUGUUGUAGUUUCUUCTG-3′)
sitbx6.2:
Sense
(SEQ ID NO: 9)
(5′-CCUGAUUUGGAUACUUCUATT-3′)
Antisense
(SEQ ID NO: 10)
(5′-UAGAAGUAUCCAAAUCAGGGT-3′)
sitbx6.3:
Sense
(SEQ ID NO: 11)
(5′-CUAGGAUCACACAGCUGAATT-3′)
Antisense
(SEQ ID NO: 12)
(5′-UUCAGCUGUGUGAUCCUAGGG-3′)

SiRNA was delivered to cells using the 96 well Amaxa nucleofector system (Lonza). Approximately 200,000 cells were nucleofected with 600 ng of siRNA with the mouse basic neuron kit (Lonza). Cells were plated at a density of 25,000 cells per well in a 96 well PDL-precoated BD Biocoat plate (BD Biosciences) coated with laminin (5 μg/ml; Invitrogen). Cells were grown overnight in N3/F12 with 25 ng/ml NGF before 20 hours of NGF deprivation by addition of NGF antibodies (25 μg/ml). Cells were fixed with PFA/sucrose and labeled for tubulin.

Automated continuous axon length measurements were taken as follows. Black walled 96 well BD Biocoat plates were imaged by an ImageXpress® Micro (Molecular Devices) with the 4× objective using laser based and image based focusing with 2× binning. The exposure time was 200 milliseconds. A single well consisted of nine images that were stitched together in MetaExpress. Each well was analyzed with the “Angiogenesis tube length” plug-in. The “tube length per set” or the equivalent of “continuous axon length” was averaged amongst 3-24 wells.

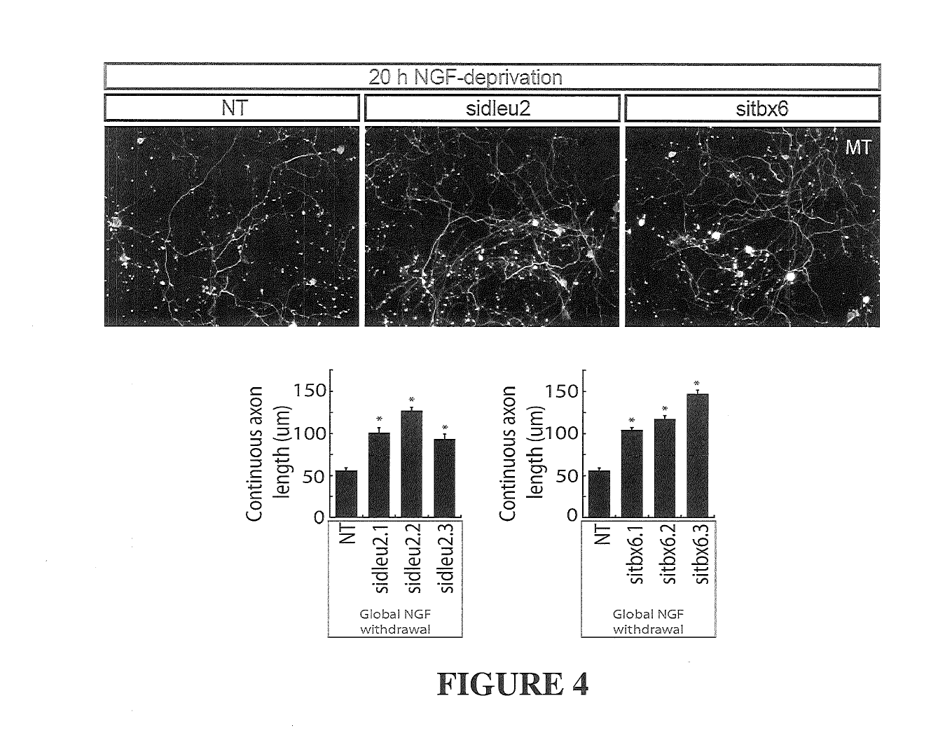

After NGF withdrawal, axons usually degenerate within 20 hours. As show in FIG. 4, the reduction of dleu2 and tbx6 expression with siRNA reduces axon degeneration following NGF withdrawal. These data implicate dleu2 and tbx6 in NGF-withdrawal induced axon degeneration.

A similar experiment was also performed in the presence of a constitutively active version of GSK3 (GSK3S9A). Overexpression of GSK3S9A results in axonal degeneration. Since tbx6 and dleu2 upregulation is dependent on GSK3, tbx6 and dleu2 knockdowns were performed to test whether the knockdown could block axonal degeneration downstream of GSK3 activation.

Briefly, hippocampus/cortical tissue was removed from E19 Sprague Dawley Rat embryos (Charles River) and dissociated after trypsin digestion. 20,000 live cells in Nbactiv4® medium (Brainbits) were plated in each well of 96 well PDL-precoated Biocoat plates (BD Biosciences). After 5 days in culture, cells were transfected with a constitutively active version of GSK3 (S9A) or an empty vector; pooled siRNA (3 siRNAs described above for tbx6 or dleu2); and GFP as a marker. After 1 and 3 days expression (6-8 DIV) cells were fixed with PFA/sucrose and labeled with GFP primary antibody (Invitrogen) followed by Alexa fluor-488 secondary (Invitrogen).

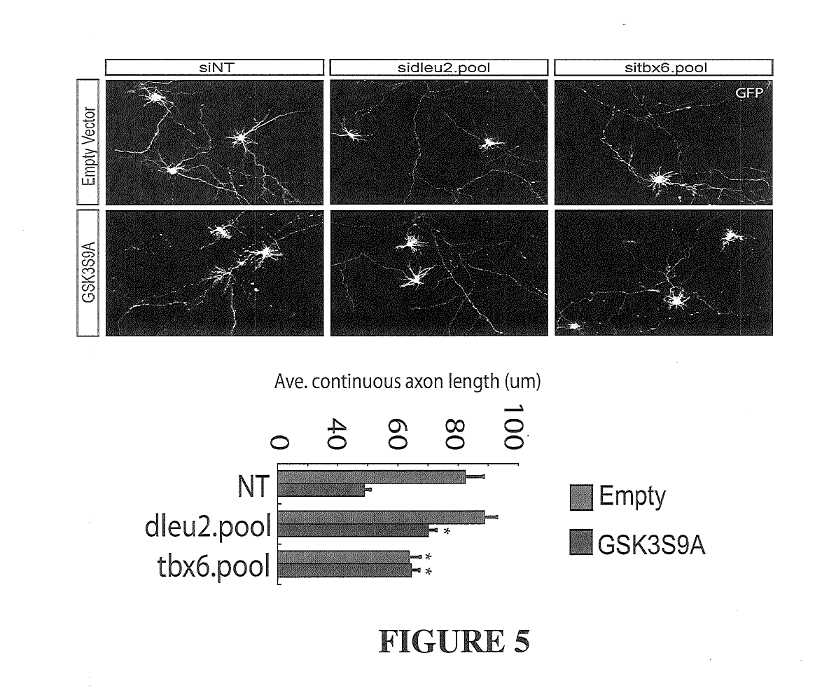

As can be seen in FIG. 5, knockdown of dleu2 and tbx6 provides protection against axonal degradation caused by GSK activation. These data provide support that GSK3 regulates a transcriptional program which includes upregulation of dleu2 and tbx6 for axon degeneration. Indeed, when axons are locally deprived of NGF for 12 hours in the presence of a p38MAP kinase inhibitor, expression of both dleu2 and tbx6 is reduced to levels similar to that observed in the presence of NGF. These data suggest that p38MAP kinase may be upstream of GSK in the axon degeneration transcriptional program that includes delu2 and tbx6.

### Example 4

### Analysis of Tbx6 and Dleu2 Gene Expression in AD and PD Patients

Two genes, tbx6 and dleu2 were identified as being overexpressed in neurons undergoing axon degeneration in a time course microarray analysis. In order to determine if the product of these genes play a role in neurodegenerative disease, brain samples from diseased patients were examined to measure expression of tbx6 and dleu2.

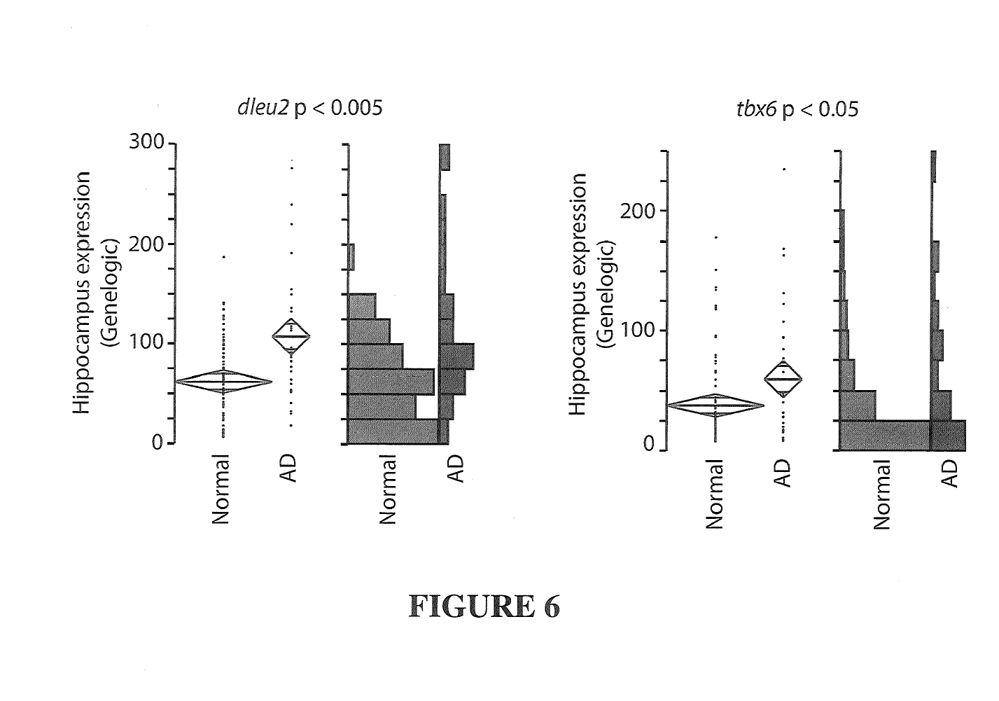

Human tbx6 and dleu2 gene expression was analyzed using a proprietary database containing gene expression information (GeneExpress®, Gene Logic Inc., Gaithersburg, Md.). Graphical analysis of the GeneExpress® database was conducted using a microarray profile viewer. FIG. 6 is a graphic representation of tbx6 and dleu2 gene expression in the hippocampus portion of the brain from human patients with AD compared to normal, non-diseased patients. The scale on the y-axis of the graph indicates gene expression levels based on hybridization signal intensity. FIG. 6 shows increased txb6 and dleu2 gene expression in diseased brain tissues relative to their normal counterparts, indicating that these genes are involved in AD and PD human disease and thus are biomarkers for AD.

What is claimed is:

1. A method of diagnosing a neurodegenerative disorder in a subject, the method comprising determining whether a subject comprises a cell that expresses at least one of the genes tbx6 and dleu2 at a level greater than the expression level of the respective genes in a reference sample, wherein presence of said cell indicates that the subject has a neurodegenerative disorder.

2. A method of monitoring disease in a subject treated for a neurodegenerative disorder, said method comprising determining whether the subject comprises a cell that expresses at least one of the genes tbx6 and dleu2 at a level greater than the expression level of the respective genes in a reference sample, wherein presence of said cell indicates that the subject is in need of continued treatment for said neurodegenerative disorder.

3. A method of assessing predisposition of a subject to develop a neurodegenerative disorder, said method comprising determining whether the subject comprises a cell that expresses at least one of the genes tbx6 and dleu2 at a level greater than the expression level of the respective genes in a reference sample, wherein presence of said cell is indicative of a predisposition for the subject to develop a neurodegenerative disorder.

4. The method of claims 1-3, wherein the cell is a neuron.

5. The method of any one of claims 1-4, wherein the method further comprises obtaining a biological sample from the subject.

6. The method of claim 5, wherein the biological sample is selected from the group consisting of cerebrospinal fluid, brain tissue, whole blood, plasma and serum.

7. The method of any one of claims 1-4, wherein determining whether a subject comprises a cell that expresses at least one of the genes tbx6 and dleu2 is performed in vivo.

8. A method of determining whether a neuron is at risk and/or is undergoing neuronal degeneration comprising determining whether the neuron expresses at least one of the genes tbx6 and dleu2 at a level greater than the expression level of the respective gene in a neuron not undergoing neuronal degeneration, wherein the increased expression of at least one of the genes tbx6 and dleu2 indicates that the neuron is at risk and/or is undergoing neuronal degeneration.

9. The method of any one of claims 1-8, wherein the expression level of at least one of the genes tbx6 and dleu2 is determined based on RNA expression, or protein expression, or a combination thereof.

10. The method of any one of claims 1-9, wherein the expression of at least one of the genes tbx6 and dleu2 is determined using a PCR method, microarray chip or a combination thereof.

11. The method of any one of claims 1-9, wherein the expression of at least one of the genes tbx6 and dleu2 genes is determined using an immunoassay.

12. The method of claim 11, wherein the immunoassay is an ELISA.

13. The method of any one of claims 1-12, wherein expression of dleu2 is measured using a PCR method, a microarray chip or a combination thereof and the expression of tbx6 is measured using an immunoassay.

14. The method of any one of claims 1-4 and 7-9, wherein the expression of at least one of the genes tbx6 and dleu2 is determined using an imaging method selected from the group consisting of magnetic resonance imaging (MRI), positron emission tomography (PET), single photon emission tomography (SPECT), x-ray computed tomography (CT), fluorescence-mediated molecular tomography (FMT), fluorescence reflectance imaging (FRI), bioluminescence imaging (BLI), gamma imaging and magnetic resonance spectroscopy.

15. The method of any one of claims 1-14, wherein the expression of both tbx6 and dleu2 is determined.

16. The method of any one of claims 1-15, wherein the neurodegenerative disorder is selected from the group consisting of Alzheimer's Disease (AD), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), Adult Onset Diabetes, senile cardiac amyloidosis, endocrine tumors, glaucoma, Alexander disease, Alper's disease, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Huntington disease, Kennedy's disease, Krabbe disease, Machado-Joseph disease (Spinocerebellar ataxia type 3), Multiple System Atrophy, Neuroborreliosis, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Charcot-Marie-Tooth disease, Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, Islets of Langerhans diabetes Type II insulinoma and other amyloidosis-related diseases.

17. The method of claim 16, wherein the neurodegenerative disorder is Alzheimer's disease (AD).

18. A kit comprising probes for detecting expression of at least one of the genes tbx6 and dleu2 and instructions for using the probes to determine whether a subject comprises a cell that expresses at least one of the genes tbx6 and dleu2 at a level greater than the expression level of the respective genes in a normal reference sample.

19. The kit of claim 18, wherein presence of said cell indicates that the subject has a neurodegenerative disorder.

20. The kit of claim 18, wherein presence of said cell indicates that the subject is in need of continued treatment for said neurodegenerative disorder.

21. The kit of claim 18, wherein presence of said cell is indicative of a predisposition for the subject to develop a neurodegenerative disorder.

22. The kit of claim 18, wherein presence of said cell is indicative that the cell is undergoing or predisposed to neurodegeneration.

23. The kit of any one of claims 18-22, wherein the cell is a neuron.

24. The kit of any one of claims 18-23, wherein the probes are labeled.

25. The kit of any one of claims 18-24, wherein the probes are selected from the group consisting of polynucleotides, antibodies or a combination thereof.

26. The kit of claim 25, wherein the antibodies are selected from the group consisting of monoclonal antibody, chimeric antibody, humanized antibody, Fv fragment, Fab fragment, Fab′ fragment, and F(ab′)₂ fragment.

27. The kit of claim 25, wherein the polynucleotide is an antisense polynucleotide.

28. The kit of claim 25, wherein the polynucleotide is a peptide nucleic acid (PNA).

29. The kit of any one of claims 18-28, wherein the probe is conjugated to a brain targeting peptide.

30. The kit of claim 29, wherein the brain targeting peptide is selected from the group consisting of insulin, transferrin, an anti-transferrin receptor antibody or fragments thereof.

31. The kit of any one of claims 18-30, wherein the neurodegenerative disorder is selected from the group consisting of Alzheimer's Disease (AD), Lewy body dementia, Down's syndrome, hereditary cerebral hemorrhage with amyloidosis (Dutch type); the Guam Parkinson-Dementia complex; as well as other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), Adult Onset Diabetes, senile cardiac amyloidosis, endocrine tumors, glaucoma, Alexander disease, Alper's disease, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Huntington disease, Kennedy's disease, Krabbe disease, Machado-Joseph disease (Spinocerebellar ataxia type 3), Multiple System Atrophy, Neuroborreliosis, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Charcot-Marie-Tooth disease, Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Gerstmann-Straussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, Islets of Langerhans diabetes Type II insulinoma and other amyloidosis-related diseases.

32. The kit of claim 31, wherein the neurodegenerative disorder is Alzheimer's disease.
